(19) United States
(12) Patent Application Publication
(10) Pub. No.: US 2019/0293527 A1
(43) Pub. Date: Sep. 26, 2019

(54) EXTRACTION OF MYCOTOXINS

(71) Applicant: Charm Sciences, Inc., Lawrence, MA (US)

(72) Inventors: JOHN JABOUR, Farmington, NH (US); Steven J. Saul, Arlington, MA (US); Mark E. Tess, Merimack, NH (US)

(73) Assignee: Charm Sciences, Inc., Lawrence, MA (US)

(21) Appl. No.: 16/176,189

(22) Filed: Oct. 31, 2018

Related U.S. Application Data
(63) Continuation of application No. 14/372,088, filed on Jul. 14, 2014.

(51) Int. Cl. G01N 1/34; G01N 33/543; G01N 33/569; G01N 33/10; G01N 1/40

(52) U.S. Cl. CPC G01N 1/34; G01N 33/54366; G01N 33/56961; G01N 2333/37; G01N 1/4055; Y10T 436/25375; G01N 2001/4061; G01N 33/10

(57) ABSTRACT

A method for extracting a mycotoxin, when present, from a sample. Compositions and methods include the use of high ionic strength compositions including compositions that include many amine and/or carboxyl groups such as protein based, amino acid based and polyethylene glycol based composition.

### REFERENCE TO PRIOR APPLICATIONS

This application is a Continuation of U.S. application Ser. No. 14/372,088, filed Jul. 14, 2014, which claims the benefit of PCT/US2013/024650, which claims the benefit of U.S. Provisional Application No. 61/594,433, filed Feb. 3, 2012 and U.S. Provisional Application No. 61/618,245, filed Mar. 30, 2012 (all of which are hereby incorporated by reference in their entireties).

### BACKGROUND

The technical field of the invention relates to methods of extracting analytes, for example mytoxins, from a sample, such as, for example, grain, or, for example, maize.

Tests to detect one or more analytes in samples are known in the art. Some examples are described in U.S. Pat. No. 5,985,675, issued Nov. 16, 1999; U.S. Pat. No. 6,319,466, issued Nov. 20, 2001; U.S. Pat. No. 7,410,808, issued Aug. 12, 2008; International Publication Number WO 2006/089027, filed Feb. 16, 2006; U.S. Pat. No. 7,785,899, issued Aug. 31, 2010; U.S. Pat. No. 7,863,057, issued Jan. 4, 2011 and International Patent Application Number PCT/US10/39113, filed Jun. 18, 2010 the teachings of all of which are incorporated herein by this reference.

When chromatographic test strips, such as lateral flow test strips, are the testing medium, many sample matrices, such as solid or granular materials, require extraction of analyte into a liquid matrix prior to testing. For example, corn can be ground and the ground sample extracted with various combinations of solvents. Typical solvents include 70% methanol in a 2:1 ratio (2 milliliter per 1 gram of sample) and acetonitrile, ethanol or other concentrations of methanol, for example at 50%, 60%, or 80%. Depending on the use, such solvents can be relatively hazardous and costly. In addition, such solvents may require further dilution or buffering prior to application to a chromatographic medium or other testing medium, such as when using certain chromatographic test strips. Dilution can affect test sensitivity and, therefore, when higher sensitivity is desired, such as in jurisdictions, such as the European Union, which require higher sensitivity to certain toxins as compared to, for example, the United States, dilution can be undesirable. The solvent also may require adjustment depending on either or both the particular matrix from which the analyte is being extracted and the particular analyte being extracted and detected. That is, one solvent may not be a “one size fits all” but, instead require adjustment/optimization depending on the matrix and/or analyte of detection. We describe herein methods and compositions for performing relatively non-hazardous extractions of a variety of toxins, such as mycotoxins, from a sample. Examples of such non-hazardous extraction compositions include a variety of high ionic strength compositions, including those with relatively abundant amine and carboxyl groups, such as protein, amino acid and polyethylene glycol based compositions.

### SUMMARY

Aspects include a method of detecting an analyte, such as a one or more mycotoxins, for example aflatoxin, ochratoxin, T2, zearalanone, vomitoxin (deoxynivalenol aka DON), patulin and fumonisin, or other of a variety of mycotoxins and other toxins in a sample. The steps of the method can include: extracting the analyte from the sample to form an extract, the extracting including contacting the sample with a composition that includes substances with high ionic strength including substances with abundant amine and/or carboxyl groups such as amino acids and a variety of proteins (the extraction composition); contacting the extract with a labeled receptor to form a mobile phase, the labeled receptor characterized by an ability to bind to the analyte to provide, in the mobile phase, a labeled receptor-analyte complex and further characterized by an ability to provide a detectable signal when the labeled receptor is captured on a solid support; contacting the mobile phase with a first test area on a solid support, the first test area comprising a first test area capture agent immobilized on the solid support, said first test area capture agent configured to both capture labeled receptor unbound by the analyte from the sample and not capture the labeled receptor-analyte complex; and measuring the intensity of the detectable signal at the first test area, wherein the intensity of the detectable signal is related to the concentration of the analyte in the sample. The extraction composition can be provided to a user in a solution or can be in a solid form such as tablet, powder or other solid forms that can be liquefied. When provided to the user in tablet or powder form, for example, with instructions to add water or other available liquid, costs are reduced by the lowering of shipping weight. In addition, longer shelf life may be achieved when maintained in non-liquid form until prior to use.

When the extraction composition is a liquid, the pH can be in the range of between pH 6 and pH 8.5. In one example a protein solution, including 2%-10%, for example, 5% protein, in buffered solution, for example 20 mM sodium phosphate at pH 7.2, was used. In another examples 0.2 M amino acid solution, for example 0.2M arginine in water, at pH 7.2 were used. Examples of proteins used in the protein solution include, alone or in combination, bovine collagen, bovine serum albumin, gelatin peptone, soy peptone, soy/casein primatone and primatone RL (TPNC). Examples of useful amino acids include, alone or in combination, arginine, glycine and arginine. In some aspects the label of the labeled receptor is a colored particle, such as a gold particle.

Aspects include a chromatographic test strip, such as a chromatographic lateral flow test strip, such as a chromatographic lateral flow test strip including nitrocellulose and/or POREX (Porex is a registered trademark of Porex Technologies Corp., Fairburn, Ga.), as a test medium, as the solid support. The test strip can include a test medium, for example a stationary phase membrane in contact or contacted with the mobile phase composition and having a first end and a second end, wherein the membrane allows lateral capillary flow of the sample from the first end to the second end and has the test areas thereon. The test strip can also include a mobile phase membrane that is the same as, or different, from the stationary phase membrane

In some aspects the labeled receptor comprises a labeled antibody, such as a polyclonal or monoclonal antibody. In other aspects the labeled receptor can be a labeled non-immunological receptor such as an enzyme. The labeled receptor can also be a combination of different receptors with differing affinities, such as differing affinities to the same analyte or affinity to different analytes.

Aspects include extraction and/or detection of one or more analytes including one or more toxins, such as mycotoxins, for example, aflatoxin, vomitoxin (DON), fumonisin, T2, zearlanone, patulin and ochratoxin from a variety of solid matrices including feeds and grains such as corn, dried distillers grain, corn gluten meal, barley, wheat and soybean.

Aspects include providing an extractant composition that is compatible with a lateral flow test strip and can effectively and efficiently extract any of one or more of a variety mycotoxins from a variety of sample matrices. Such aspects include providing a standard extractant composition that can be used, with little or no alteration or adjustment, across a variety of matrices, analytes and/or tests.

Aspects include a composition that can both extract an analyte of interest and efficiently and effectively flow on a lateral flow test strip. Such aspects can include a composition that can both block binding sites on a chromatographic test strip membrane such as a nitrocellulose membrane and/or a POREX membrane and extract an analyte of interest. Such compositions can include, for example, bovine serum albumin (BSA) and/or polyethylene glycol (PEG). PEG based compositions can also be usefully combined with other materials such as other buffers, for example, POPSO (Piperazine-1,4-bis(2-hydroxy-3-propanesulfonic acid).

Aspects include providing an extraction composition that can be used to extract any of a number of toxins, such as mycotoxins, from any of a number of matrices, such as feed and grains, with little or no dilution or buffering prior to addition to a lateral flow test strip. Such aspects can include an extraction composition that also serves as an agent to block binding cites on a lateral flow test strip membrane, for example nitrocellulose.

Aspects include providing an extraction composition that can be used to extract any of a number of toxins, such as mycotoxins, from any of a number of matrices, such as feed and grains, with little or no dilution or buffering prior to addition to a lateral flow test strip. Such aspects can include an extraction composition that also serves as an agent to block binding cites on a lateral flow test strip membrane, for example nitrocellulose.

Aspects include providing an extraction composition that can be used extract one or more toxins from a sample for testing to detect one or more toxins in a single test such as a single lateral flow test ship.

### DETAILED DESCRIPTION

Provided are compositions and methods for facilitating the extraction of small molecules, such as mycotoxins, from agricultural products such as a variety of grains, corns and feeds. These compositions and methods include the use of materials with high ionic strength such as materials having one or multiple amine groups including a variety of protein and amino acid based compositions and materials. Useful compositions can include one or more proteins and/or amino acids. A variety of proteins can be usefully employed including alone or in combination bovine collagen, bovine serum albumin (BSA), gelatin peptone, soy peptone, soy/casein primatone and primatone RL (TPNC). BSA can be particularly useful since it is relatively inexpensive, readily available and compatible with many lateral flow test strips. For example, BSA is useful to block binding sites on nitrocellulose when nitrocellulose is a component of a test strip. In addition, BSA can be useful to enhance the flow along a test strip. When milk is the matrix, the casein in the milk can block binding sites on nitrocellulose and, therefore, the importance of utilizing other blocking agents may be reduced. When milk, or other matrices that include agents that block binding sites on chromatographic membranes, are not tested, the sites may need to be blocked and, therefore, BSA can be useful. The properties of BSA allow a BSA based solution to be used on a test strip with little or no further dilution. For that reason, when BSA is used as an extractant, higher sensitivity to analyte may be achievable.

Proteins, such as BSA, can be used in a mixture, for example including a salt such as a phosphate salt, citrate salt and/or chloride salt. Other possibly useful ingredients include certain wetting agents, chelators and preservatives.

An extraction can take place using a variety of methods including shaking the sample in a container, mixing the sample with a stirrer, or mixing the sample with a blender. Depending on the composition, an antifoaming agent may be useful. Other possible extraction include filtering to collect the extract, allowing sample to sit to form an extract layer above the ground sample, or centrifuging a portion of the sample to obtain an extract layer and sample layer.

The above described extraction methods can be useful to extract an analyte for detection in for a variety of detection methods and have been found particularly useful for extraction of one or more small molecules, such as one or more mycotoxins, in preparation for detection in lateral flow test strips. For lateral flow test strips, the sample extract can be tested directly or can be mixed with a dilution buffer. The dilution buffer can be used to allow a mobile phase to flow uniformly over the test strip. A mobile phase allows reconstitution of the dried reagents on the test strip. The extract can be diluted by a number of methods and a variety of possible dilution ratios of the extract with the dilution buffer. The dilution buffer can consist of, for example, phosphate buffer, or water. When the analyte is in sample liquid, such as fluid milk, the sample may not require dilution or extraction. When the sample is a solid, or semi-solid, and, therefore, must be combined with a liquid for test operation, the use of a composition for extraction, such as BSA and/or PEG based compositions, which can be added directly to a test strip without further dilution, is useful. Combinations with other materials, however, may nevertheless be desirable such as to alter the test sensitivity range or to allow consistency between samples. Similarly, for example water soluble analytes, such as DON, may be extracted from samples using a composition that is partially or completely water. Such analyte, however, may still require to be mixed with a composition, such as a 5% BSA composition, prior to addition to a chromatographic test strip to block binding sites such as nitrocellulose binding sites, which might otherwise interfere with the operation.

The lateral flow assay can include a support strip and a sample-absorbing matrix. The test device also can include a mobile-phase support attached to the support strip and in contact with the sample-absorbing matrix. In an example, a mobile-phase composition is disposed within or on the test device and has one or more labeled receptors, such as one or more gold labeled antibodies.

The mobile phase composition can be applied prior to test operation, for example by spraying and drying onto a porous surface such as a polyethylene membrane. When exposed to a sample, the mobile-phase composition can be carried in the sample flow together with the sample. In test operation, the sample flows and, when a receptor is an antibody, the antibody binds to an analyte present in the sample to form an antibody-analyte complex. Alternatively, the mobile phase can be combined with sample prior to application to the test strip or other solid support. In this alternative embodiment, antibody can bind to analyte in the sample prior to contact with the test strip.

In an example, the test strip includes a stationary-phase support strip, which may be part of the same strip as the mobile-phase composition support strip, or on a separate strip in fluid flow contact with the first strip. A support strip can have a first membrane end in contact with the mobile-phase composition and a second membrane end that may be in contact with an optional disposal zone. Lateral-capillary flow of the sample is from the first membrane end to the second membrane end. The test strip can also be wholly or partially of a material, for example nitrocellulose, that can bind proteins. A variety of materials can be used in various portions of the strip including natural or synthetic materials including cellulosic materials such as paper, cellulose and cellulose derivatives such as cellulose acetate and nitrocellulose; fiberglass; glass fiber filter, for example WHATMAN Fusion 5 membrane (Whatman is a registered trademark of Whatman Paper Limited, Kent, England); cloth, both naturally occurring and synthetic; porous gels such as silica gel, agarose, dextran and gelatin; porous fibrous matrices; starch based materials, such as cross-linked dextran chains; ceramic materials; films of polyvinyl chloride and combinations of polyvinyl chloride-silica; POREX (Porex is a registered trademark of Porex Technologies Corp., Fairburn, Ga.) and the like. Generally, the material used in the flow stream should allow liquid to flow on or through the strip. If a variety of materials are used they can be in fluid flow communication/contact or capable of being brought into fluid flow communication/contact. The strip should have sufficient inherent strength or additional strength can be provided by a supplemental support such as a plastic backing upon which porous or bibulous strip components are attached.

One or more test zones can be located on the test strip and may include a capture agent, such as a representative analyte or analogue thereof, which captures unbound labeled receptor, such as unbound labeled antibody. Examples of possible test zone capture agents include aflatoxin, or other toxins such as ochratoxin, DON, T2, patulin, zearlanone and fumonisin, depending on the analyte to be detected. Such a capture agent may be disposed on the test zone portion of the membrane for example by spraying. Prior to spraying, said capture agent can be conjugated to an attachment or carrier protein. Suitable attachment proteins are known to those skilled in the art to be proteins that bind readily to solid supports, such supports that include nitrocellulose. A useful attachment protein includes a carrier protein, i.e., a protein commonly used in conjunction with an immunogen, such as generally water soluble proteins with multiple accessible amino groups including albumin, e.g., bovine serum albumin (BSA), ovalbumin (OVA), keyhole limpet hemocyanin (KLH) and thyroglobulin (THG).

One or more optional control zones may also be on the test strip. The control zone may contain capture agent for the analyte receptor, such as an antibody with affinity to the analyte. Such capture agent can include antibody to the particular antibody, such as anti-species antibody, for binding with both analyte-bound antibody and excess unbound antibody. Alternatively, the control zone may be involved in an independent reaction that informs the user that the test is complete and includes consistent visual indicators, such as color development, for comparison to the test zone. The control zone can generate signal either on contact with sample or on contact with specific test material, such as labeled antibody, such as when the control zone includes an anti-species antibody or one of the several useful antibody capture agents known in the art including protein A, protein G or recombinant varieties of proteins A and G.

The lateral flow test device and method can also be in a sandwich assay format or, as described above, an inhibition/competitive format.

Lateral flow test results can be interpreted visually or by use of a reader, or analyzer, such as a ROSA® reader (ROSA is a registered trademark of Charm Sciences, Inc. Lawrence, Mass.). Other reader/analyzer examples include fluorometers, luminometers, bar code readers, radiation detectors (such as scintillation counters), UV detectors, infrared detectors, electrochemical detectors or optical readers, such as spectrophotometers. The reader can be used to distinguish between one or more test zones and one or more control zones or simply to determine a relative change in the test zone. In one embodiment the reader is a ROSA reader. In a particular embodiment, the analyzer is an optical reader, e.g., the reader described in U.S. Pat. No. 6,124,585, issued Sep. 26, 2000, hereby incorporated by reference. In a quantitative test, the changes in the test areas, and, when a control zone is present, the extent of the difference between the control zone and test zone or test areas (test area and test zone are used interchangeably herein), can determine the test range detection level of analyte. To accurately and/or numerically assess the differences and the binding at the control zone and test zone, particularly in a quantitative assay, a reader is useful. The reader can also include, within its settings, various selectable calibration settings. Such calibration settings can be editable or changeable depending on the matrix being test and/or the analyte being detected. In that way, for example, a standard curve can be adjusted to reflect the efficiency of extraction of a particular analyte from a particular matrix. Such an adjustable reader can be particularly useful to allow standardization of a surfactant based extraction solution, such as described herein, for use with a variety of matrices and a variety of analytes.

In a particular embodiment, the mobile phase contacts, or is put into contact with, a first test area on a solid support. The solid support can be configured to allow the mobile phase to flow from the first test area to a second test area on the solid support and, if a control zone is included, to the control zone. The first test area can include a capture agent immobilized on the solid support. The first test area capture agent will have greater binding affinity to the receptor than to the receptor-analyte complex. As a result of that differential in binding affinity, captured receptor in the test area will decrease as sample analyte concentration increases. When there is a second test area, the second test area can also include a capture agent immobilized on the solid support. As with the first area capture agent, the second test area capture agent will have greater binding affinity to the receptor than to the receptor-analyte complex. The capture agent can be the same in each of the test areas and at the same or different concentrations in each area. The capture agents can also be different, for example with different binding characteristics to the receptor. The capture agents in different test areas can also be targeted to entirely separate receptors, such as when the test strip is designed to detect multiple analytes.

The receptor can be labeled with a label, such as a colored particle, that can be detected when the receptor is bound to the solid support via capture by the capture agent immobilized on the solid support. The intensity of the detectable signal, for example a visible signal, at the first and second test areas can be measured to determine a result. In an inhibition style test the strength (intensity) of the signals are inversely related to the concentration of analyte in the sample. The signal intensities can be observed visually or measured by an electronic test instrument. For example the intensity at each of the two test areas can be summed to determine a result that can relate to the concentration of an analyte in the sample.

Various suitable labels include chromogens, catalysts, fluorescent compounds, chemiluminescent compounds, radioactive labels, magnetic beads or magnetic particles, enzymes or substrates, vesicles containing signal producing substances, colorimetric labels, direct visual labels including colloidal metallic and metallic and non-metallic colored particles, dye particles, or organic polymer latex colored particles.

Presence or absence tests, known in the art as qualitative tests, provide a yes or no result. Tests that detect the presence or absence of a target analyte above or below a certain threshold level are known as semi-quantitative tests. Tests that determine that a target analyte is present at a particular concentration, or within a range of concentrations, are known as quantitative tests.

Although, many of the herein examples and descriptions refer to detecting mycotoxins such as aflatoxin, zearalanone, patulin, DON, fumonisin and ochratoxin, other analytcs can be detected and quantified in a variety of matrices using the herein disclosure. Other possible target analytes include hormones, vitamins, drugs, metabolites and their receptors and binding materials, antibodies, peptides, protein, allergens, fungicides, herbicides, pesticides and plant, animal and microbial toxins may be determined using the present methods and apparatuses. Other analytes that may be determinable by this disclosure include antibiotics, such as beta-lactams, cephalosporins, erythromycin, sulfonamides, tetracyclines, nitrofurans, quinolones, vancomycin, gentamicin, amikacin, chloramphenicol, streptomycin and tobramycin, toxins, and drugs of abuse, such as opioids and the like, as well as the metabolites of any of the above listed possible target analytes.

Although much of the description herein relates to use of the extraction compositions for extracting analytes for detection using lateral flow type devices and tests, it will be appreciated that the extraction compositions described herein may also be useful to extract analytes, such as toxins, prior to detection in other test formats, for example ELISA assays, radiobinding assays such as those available from Charm Sciences, Inc. and known as the Charm II assays, and other detection methods and tests.

Numerous embodiments and advantages of this disclosure have been set forth in the foregoing description. Many of the novel features are captured in the following claims. The disclosure, however, is illustrative only, and modifications by one of skill in the art may be made with the present disclosure without departing from the scope of the invention.

### Examples

Within the tables, T1 is test line 1 result; T2 is test line 2 result; C is Control line result; RR Cone is the concentration provided by the ROSA Reader (using a preprogrammed algorithm); Result is Rosa Reader result. The ROSA Reader is programmed to provide a result and RR concentration. The result is calculated from a comparison of T1 and T2 with C using an algorithm. The “spread” is the difference between the result for a negative control (NC) result and the result with the particular analyte concentration. The RR Cone is determined by the ROSA Reader through a calculation that associates the Result with a concentration for the particular toxin and matrix. In Tables 1-3 the ROSA reader was calibrated using results from extractions using 70% methanol and, therefore, RR Cone is relevant. For tables 4-14, the RR Cone results are not calibrated and, therefore, sever only as a relatative indicator of detection (the concentration is not accurate becase the reader is not caibrated).

Table 1 results are from an experiment using a 70% methanol extraction solution. The original sample was a 1000 ppb fumonisin B1, B2, and B3 in corn and the same sample was diluted to an in-assay concentration of 10.6 ppb. % cross-reactivity is a ratio of the RR Cone for a cross-reacting analyte (in Tables 1-3 fumonisin B2 and B3) with the RR Cone for B1. A lower cross-reactivity percentage indicates greater specificity to the analyte of detection, which in Tables 1-3 is fumonisin B1. It can be desirable to have cross-reactivity, such as when detection of the cross-reacting substances is desired. Cross-reactivity is, however, undesirable when the cross-reacting substance is not to be detected and, therefore, is a test interference. Results are in parts per trillion. For example, the RR Cone result in Table 1, for in-assay 10.6 ppb B1 is a RR Cone for the sample of 0.933 ppm, or 933 parts per billion.

TABLE 1

| | | | | | BR | |
|---|---|---|---|---|---|---|
| | | T1 | T2 | C | Conc | Result |
| Conc | | 2710 | 4513 | 2445 | 0.9 | −2333 |
| 10.6 ppb B1 | | 3107 | 3451 | 2223 | 1 | −2112 |
| | | 2672 | 4027 | 2176 | 0.9 | −2347 |
| | AVE | 2830 | 3997 | 2281 | 0.933 | −2264 |
| | % CV | 9% | 13% | 6% | 6% | 6% |
| Conc | | 4581 | 4951 | 2380 | 0.2 | −4772 |
| 10.6 ppb B2 | | 4166 | 4196 | 2074 | 0.3 | −4214 |
| | | 3543 | 4505 | 2123 | 0.4 | −3802 |
| | AVE | 4097 | 4551 | 2192 | 0.300 | −4263 |
| | % CV | 13% | 8% | 7% | 33% | 11% |
| % Cross reactive, | | 3548 | 4735 | 2485 | 0.55 | −3313 |
| B1 = 100% | | 3408 | 4734 | 2673 | 0.75 | −2796 |
| 10.6 ppb B3 | | 3211 | 4834 | 2293 | 0.5 | −3459 |
| | AVE | 3389 | 4768 | 2484 | 0.600 | −3189 |
| | % CV | 5% | 1% | 8% | 22% | 11% |
| % Cross reactive, | | | | | 64% | |
| B1 = 100% | | | | | | |

Table 2 results are from an experiment using a 5% BSA extraction solution. The original sample was 1000 ppb and the sample was diluted to an in-assay concentration of 22.7 ppb. Results show higher RR Cone as is appropriate given the higher concentration (22.7 ppb) and similar cross-reactivity.

TABLE 2

| Cone | | T1 | T2 | C | RR Cone | Result |
|---|---|---|---|---|---|---|
| 22.7 ppb B1 | | 1990 | 4016 | 2936 | 2.3 | −134 |
| | | 1924 | 3323 | 2641 | 2.5 | 35 |
| | | 1875 | 3379 | 2700 | 2.6 | 146 |
| | AVE | 1930 | 3573 | 2759 | 2.467 | 16 |
| | % CV | 3% | 11% | 6% | 6% | |
| 22.7 ppb B2 | | 2988 | 4282 | 2635 | 1 | −2000 |
| | | 2827 | 4077 | 2508 | 1.1 | −1888 |
| | | 2744 | 4482 | 2558 | 1 | −2110 |
| | AVE | 2853 | 4280 | 2567 | 1.033 | −1999 |
| | % CV | 4% | 5% | 2% | 6% | 6% |
| % Cross | | | | | 42% | |
| reactive, | | | | | | |
| B1 = 100% | | | | | | |
| 22.7 ppb B1 | | 2554 | 3893 | 2986 | 2 | −475 |
| | | 2757 | 3990 | 2846 | 1.6 | −1055 |
| | | 2279 | 3770 | 2581 | 1.7 | −887 |
| | AVE | 2530 | 3884 | 2804 | 1.767 | −806 |
| | % CV | 9% | 3% | 7% | 12% | 37% |
| % Cross | | | | | 72% | |
| reactive, | | | | | | |
| B1 = 100% | | | | | | |

Table 3 results are from an experiment using a 5% BSA extraction solution. The original sample was 100 ppb and the sample was diluted to an in-assay concentration of 10.6 ppb. Results show similar detection levels as with the methanol extraction at 10.6 ppb and similar cross-reactivity.

TABLE 3

| Cone | | T1 | T2 | C | RR Cone | Result |
|---|---|---|---|---|---|---|
| 10.6 ppb B1 | | 3018 | 5064 | 2961 | 1 | −2160 |
| | | 2847 | 3393 | 2071 | 1 | −2098 |
| | | 3375 | 3562 | 2404 | 1 | −2129 |
| | AVE | 3080 | 4006 | 2479 | 1.000 | −2129 |
| | % CV | 9% | 23% | 18% | 0% | 1% |
| 10.6 ppb B2 | | 3242 | 4743 | 2194 | 0.45 | −3597 |
| | | 3361 | 4650 | 1832 | 0.3 | −4347 |
| | | 3286 | 4558 | 2085 | 0.45 | −3704 |
| | AVE | 3296 | 4660 | 2037 | 0.40 | −3883 |
| | % CV | 2% | 2% | 9% | 22% | 10% |
| % Cross | | | | | 40% | |
| reactive, | | | | | | |
| B1 = 100% | | | | | | |
| 10.6 ppb B3 | | 2921 | 4029 | 2339 | 0.95 | −2272 |
| | | 3123 | 4379 | 2364 | 0.75 | −2774 |
| | | 3251 | 4678 | 2234 | 0.5 | −3461 |
| | AVE | 3098 | 4362 | 2312 | 0.733 | −2836 |
| | % CV | 5% | 7% | 3% | 31% | 21% |
| % Cross | | | | | 73% | |
| Reactive, | | | | | | |
| B1 = 100% | | | | | | |

Table 4-14 include results from tests using a variety of extraction compositions that include a variety of protein and amino acid based extraction solutions as indicated within each table. All protein solution were 5% protein in 20 mM NaPO4 at pH 7.2 Amino acid and other solution are 0.2M. NC results are for a negative control. ND results are for samples with zero detected aflatoxin in a sample by a reference method (depending on the limit of detection of the reference method it is possible some aflatoxin is present in ND samples). The data in table 4 shows a “spread” of 6391 between the NC result (in some cases, as in table 4, the average of two NC results) and result with a sample originally spiked at 90 ppb aflatoxin which was diluted to an in-assay contraction of approximately 5.2 ppb (90/4/4.3).

TABLE 4
5% Primatone RL, (TPNC)

| NC | T1 | T2 | C | Result |
|---|---|---|---|---|
| | 3934 | 4154 | 2318 | −3452 |
| | 3698 | 4078 | 2022 | −3732 |
| AVE | 3816 | 4116 | 2170 | −3592 |
| ND | 3733 | 4049 | 2005 | −3772 |
| 5.2 ppb | 1078 | 2397 | 3137 | 2779 |
| Spread | | | | 6391 |

TABLE 5
5% Bovine Collagen

| NC | T1 | T2 | C | Result |
|---|---|---|---|---|
| | 4614 | 4526 | 2357 | −4426 |
| | 4194 | 4430 | 2325 | −3974 |
| AVE | 4404 | 4478 | 2341 | −4200 |
| ND | 4436 | 4709 | 2401 | −4343 |
| 5.2 ppb | 738 | 2163 | 3936 | 4971 |
| Spread | | | | 9171 |

TABLE 6
85% Casein, 15% Soy

| | T1 | T2 | C | Result |
|---|---|---|---|---|
| NC | 3847 | 4399 | 2571 | −3104 |
| ND | 3575 | 3809 | 1917 | −4343 |
| 5.2 ppb | 957 | 2471 | 3503 | 3578 |
| Spread | | | | 6682 |

TABLE 7
5% Gelatin Peptone

| | T1 | T2 | C | Result |
|---|---|---|---|---|
| NC | 4494 | 4555 | 2072 | −4905 |
| ND | 4066 | 3990 | 2011 | −4034 |
| 5.2 ppb | 1400 | 2947 | 3815 | 3283 |
| Spread | | | | 8188 |

TABLE 8
5% Soy Peptone

| | T1 | T2 | C | Result |
|---|---|---|---|---|
| NC | 4419 | 4159 | 2176 | −4226 |
| ND | 4263 | 4224 | 2171 | −4145 |
| 5.2 ppb | 1176 | 2460 | 3183 | 2730 |
| Spread | | | | 6956 |

TABLE 9
0.2M Glycine pH 7.2

| | T1 | T2 | C | Result |
|---|---|---|---|---|
| NC | 4337 | 4510 | 2059 | −4729 |
| ND | 3820 | 4151 | 2232 | −3507 |
| 5.2 ppb | 1234 | 2566 | 3157 | 2415 |
| Spread | | | | 7243 |

TABLE 10
0.2M Arginine pH 7.2

| | T1 | T2 | C | Result |
|---|---|---|---|---|
| NC | 4088 | 4155 | 1799 | −4645 |
| ND | 3901 | 4115 | 1812 | −4392 |
| 5.2 ppb | 664 | 1945 | 3091 | 3573 |
| Spread | | | | 8218 |

TABLE 11
0.2M Diaminopropanc pH 7.2

| | T1 | T2 | C | Result |
|---|---|---|---|---|
| NC | 3707 | 4062 | 2239 | −3291 |
| ND | 3791 | 4043 | 2311 | −3212 |
| 5.2 ppb | 1014 | 2488 | 3272 | 3042 |
| Spread | | | | 6333 |

TABLE 12
0.2M Na Phos pH 7.2

| | T1 | T2 | C | Result |
|---|---|---|---|---|
| NC | 4294 | 4218 | 2205 | −4102 |
| ND | 4121 | 4077 | 2306 | −3586 |
| 5.2 ppb | 1727 | 3038 | 3838 | 2911 |
| 5.2 ppb | 1590 | 2784 | 3390 | 2406 |
| AVE | 1659 | 2911 | 3614 | 2659 |
| Spread | | | | 6761 |

TABLE 13
0.02M NA PO4 pH 7.2

| | T1 | T2 | C | Result |
|---|---|---|---|---|
| NC | 3072 | 3551 | 1801 | −3021 |
| 5.2 ppb | 1579 | 2648 | 3109 | 1991 |
| Spread | | | | 5012 |

TABLE 14
0.02M Arginine pH 7.2

| | T1 | T2 | C | Result |
|---|---|---|---|---|
| NC | 3883 | 4136 | 1960 | −4099 |
| ND | 3994 | 4210 | 2029 | −4146 |
| 5.2 ppb | 1401 | 2689 | 3143 | 2196 |
| Spread | | | | 6259 |

Tables 15 and 16 include results from tests using a BSA based extraction composition. The composition included 5% protein (BSA) in 0.1 M NaPO4 at pH 7.4 and 0.08% KATHON. Table 15 in-assay concentrations of aflatoxin in the sample are at 0, 2.16, 5.6, 11, 19.5, 90.1 parts per billion (PPB) (concentrations shown on far left of table). As can be seen, the difference between the results at 0 ppb and the various concentrations (the spread) grows as the concentration of aflatoxin in the sample is increased. Table 16 results are from a test sample that was diluted 4.3 fold. As a result, the in-assay concentrations are affectivity decreased by 4.3 from the concentration shown on the left of the table (for example 90.1 is actually an in-test concentrations of 20.95 (90.1/4.3).

TABLE 15

| | 1x | T1 | T2 | C | RR Cone | Result |
|---|---|---|---|---|---|---|
| 0 | | 3886 | 3773 | 2359 | 0 | −2941 |
| | | 3785 | 3082 | 1844 | 0 | −3179 |
| | | 3421 | 3323 | 2002 | 1 | −2740 |
| | | 3862 | 3919 | 2431 | 0 | −2919 |
| | AVE | 3739 | 3524 | 2159 | 0 | −2945 |
| | % CV | 6% | 11% | 13% | 200% | 6% |
| 2.16 | | 2688 | 2714 | 2542 | 7 | −318 |
| | | 2920 | 2589 | 2365 | 5 | −779 |
| | | 3266 | 2825 | 2715 | 5 | −661 |
| | | 3254 | 3204 | 2944 | 6 | −570 |
| | AVE | 3032 | 2833 | 2642 | 6 | −582 |
| | % CV | 9% | 9% | 9% | 17% | 34% |
| 5.6 | | 1984 | 2408 | 2847 | 19 | 1302 |
| | | 2124 | 2297 | 2730 | 16 | 1037 |
| | | 2232 | 2703 | 2989 | 16 | 1043 |
| | | 2357 | 2540 | 3150 | 20 | 1403 |
| | AVE | 2174 | 2487 | 2929 | 18 | 1197 |
| | % CV | 7% | 7% | 6% | 12% | 15% |
| 11 | | 915 | 1642 | 2750 | 50 | 2943 |
| | | 1459 | 1956 | 2945 | 30 | 2475 |
| | | 1329 | 2144 | 3065 | 42 | 2657 |
| | | 993 | 1662 | 2827 | 52 | 2999 |
| | AVE | 1174 | 1851 | 2897 | 44 | 2769 |
| | % CV | 22% | 13% | 5% | 23% | 9% |
| 19.5 | | 972 | 1644 | 3038 | 67 | 3460 |
| | | 1007 | 1752 | 3055 | 63 | 3351 |
| | | 755 | 1438 | 2505 | 47 | 2817 |
| | | 799 | 1440 | 2775 | 62 | 3311 |
| | AVE | 883 | 1569 | 2843 | 60 | 3235 |
| | % CV | 14% | 10% | 9% | 15% | 9% |
| 90.1 | | 289 | 950 | 3643 | 150 | 6047 |
| | | 251 | 964 | 3633 | 150 | 6051 |
| | | 88 | 734 | 3145 | 150 | 5468 |
| | | 136 | 908 | 3678 | 150 | 6312 |
| | AVE | 191 | 889 | 3525 | 150 | 5970 |
| | % CV | 50% | 12% | 7% | 0% | 6% |

TABLE 16

| | | 4.3x | | | | |
|---|---|---|---|---|---|---|
| Cone | | T1 | T2 | C | RR Cone | Result |
| 0 | | 3778 | 4268 | 1923 | 0 | −4200 |
| | | 4050 | 4229 | 2122 | 0 | −4035 |
| | | 3858 | 3715 | 1923 | 0 | −3727 |
| | | 4002 | 4287 | 2302 | 0 | −3685 |
| | AVE | 3922 | 4125 | 2068 | 0 | −3912 |
| | % CV | 3% | 7% | 9% | 0% | 6% |
| 2.16 | | 4094 | 4378 | 2494 | 0 | −3484 |
| | | 4139 | 4679 | 2726 | 0 | −3366 |
| | | 4128 | 4498 | 2637 | 0 | −3352 |
| | | 3524 | 4132 | 2357 | 0 | −2942 |
| | AVE | 3971 | 4422 | 2554 | 0 | −3286 |
| | % CV | 8% | 5% | 6% | 0% | 7% |
| 5.6 | | 3745 | 4267 | 2665 | 1 | −2682 |
| | | 3819 | 3614 | 2338 | 1 | −2757 |
| | | 4005 | 4250 | 2723 | 1 | −2809 |
| | | 3809 | 4268 | 2641 | 1 | −2795 |
| | AVE | 3845 | 4100 | 2592 | 1 | −2761 |
| | % CV | 3% | 8% | 7% | 0% | 2% |
| 11 | 5 | 2743 | 3114 | 2495 | 5 | −867 |
| | 5 | 2811 | 3647 | 2691 | 4 | −1076 |
| | 5 | 3664 | 4027 | 3018 | 2 | −1655 |
| | 5 | 3157 | 3573 | 2895 | 4 | −940 |
| | AVE | 3094 | 3590 | 2775 | 4 | −1135 |
| | % CV | 14% | 10% | 8% | 34% | 32% |
| 19.5 | 5 | 2715 | 3672 | 3572 | 14 | 757 |
| | 5 | 2837 | 3810 | 3540 | 11 | 433 |
| | 5 | 2475 | 3281 | 3312 | 15 | 868 |
| | 5 | 2080 | 3053 | 3015 | 15 | 897 |
| | AVE | 2527 | 3454 | 3360 | 14 | 739 |
| | % CV | 13% | 10% | 8% | 14% | 29% |
| 90.1 | 5 | 700 | 1736 | 3815 | 150 | 5194 |
| | 5 | 616 | 1539 | 3342 | 124 | 4529 |
| | 5 | 832 | 1967 | 3977 | 150 | 5155 |
| | 5 | 684 | 1824 | 3708 | 150 | 4908 |
| | AVE | 708 | 1767 | 3711 | 144 | 4947 |
| | % CV | 13% | 10% | 7% | 9% | 6% |

What is claimed is:

1. A method for extracting one or more mycotoxin, when present, from a dry test sample comprising:
a) mixing the sample with a composition comprising a proteinaceous material in water to form an admixture;
b) providing conditions for the admixture to separate into a settled layer and a water layer; and
c) collecting at least a portion of said water layer, wherein said at least a portion of said water layer is an extract containing said one or more mycotoxins.

2. The method of claim 1, wherein, in said mixing step, said composition comprising a proteinaceous material is dry and is mixed with the sample in water to form the admixture.

3. The method of claim 1, wherein the proteinaceous material is an amino acid.

4. The method of claim 1, wherein the proteinaceous material is an albumin.

5. The method of claim 4, wherein said albumin is bovine serum albumin.

6. The method of claim 4, wherein said albumin is porcine albumin.

7. The method claim 1, wherein the proteinaceous material is Primatone RL.

8. The method of claim 1, wherein the proteinaceous material is a collagen.

9. The method of claim 1, wherein the proteinaceous material is a peptone.

10. The method of claim 9, wherein the proteinaceous material is gelatin peptone.

11. The method of claim 9, wherein the proteinaceous material is soy peptone.

12. The method of claim 1, wherein the test sample comprises a grain.

13. The method of claim 1, wherein the composition comprising the proteinaceous material further comprises a preservative.

14. The method of claim 1, wherein the composition comprising the proteinaceous material further comprises a biocide comprising 1.15% methylchloroisothiazolinone, 0.35% methylisothiazolinone, 23.00% MgCl₂ and Mg(NO₃)₂ in water.

15. The method of claim 1, wherein the composition comprising the proteinaceous material further comprises a salt.

16. A method for extracting a mycotoxin, when present, from a grain sample comprising:
a) mixing the sample with a composition comprising a proteinaceous material in water to form an admixture;
b) providing conditions for the admixture to separate into a settled layer and a water layer; and
c) collecting at least a portion of said water layer, wherein said at least a portion of said water layer is an extract containing said mycotoxin.

17. The method of claim 16, wherein the proteinaceous material is an albumin.

18. The method of claim 17, wherein said albumin is bovine serum albumin.

19. The method of claim 16, wherein the composition comprising the proteinaceous material further comprises a preservative.

20. The method of claim 16, wherein the composition comprising the proteinaceous material further comprises a salt.
